(19) United States
(12) Patent Application Publication
(10) Pub. No.: US 2025/0387011 A1
(43) Pub. Date: Dec. 25, 2025

(54) MEDICAL DEVICES AND SYSTEMS WITH SENSORS AND RELATED METHODS

(71) Applicant: Boston Scientific Scimed, Inc., Maple Grove, MN (US)

(72) Inventors: Nathan GAWORSKI, St. Paul, MN (US); Adam TRACY, Vadnais Heights, MN (US); Eric T. GAGNER, Minnetonka, MN (US); Taylor James GARCIA, St. Paul, MN (US); Steven J. MEYER, Lake Elmo, MN (US); Nathaniel BEHNING, Faribault, MN (US); Sarah Melissa GRUBA, Vadnais Heights, MN (US); Bradley S. SWEHLA, Eagan, MN (US); Daniel J. FOSTER, Lino Lakes, MN (US)

(73) Assignee: Boston Scientific Scimed, Inc., Maple Grove, MN (US)

(21) Appl. No.: 19/247,222

(22) Filed: Jun. 24, 2025

Related U.S. Application Data
(60) Provisional application No. 63/663,852, filed on Jun. 25, 2024.

(51) Int. Cl. A61B 1/00

(52) U.S. Cl. CPC A61B 1/00097; A61B 1/00114; A61B 2560/0462; A61B 2562/0247; A61B 2562/0271; A61B 2562/028; A61B 2562/227

(57) ABSTRACT

Medical devices and systems having a micro-electromechanical system (MEMS) assembly are described herein. The medical device may include a shaft extending to a distal end and a pressure sensor assembly incorporated into the shaft. The pressure sensor assembly may include a housing, a MEMS chip, and a material. The housing may define a chamber and include a first body coupled to a second body, e.g., the first body defining at least one opening in fluid communication with the chamber. The MEMS chip may include a diaphragm. The material may at least partially cover the diaphragm and have an elasticity or viscosity that permits transfer of force therethrough. The pressure sensor assembly may be configured to measure pressure external to the medical device.

### CROSS-REFERENCE TO RELATED APPLICATIONS

This application claims the benefit of priority to U.S. Provisional Application No. 63/663,852, filed on Jun. 25, 2024, which is incorporated by reference herein in its entirety.

### TECHNICAL FIELD

Aspects of the present disclosure generally relate to medical assemblies, devices, and systems. In particular, some aspects relate to medical devices and systems having a micro-electromechanical system assembly incorporated in a distal portion of the medical device.

### BACKGROUND

Medical devices are often inserted into the body to perform a therapeutic and/or diagnostic procedure inside a subject's body. An example of such a device is an ureteroscope or other type of scope, which includes an insertion portion that is introduced into the body. Various features of the scope may assist in performing a therapeutic and/or diagnostic procedure inside the subject's body. Sensors and other electronic components may be susceptible to damage, e.g., due to external forces. Such forces can degrade performance over time or ultimately lead to failure of the electronic component(s).

### SUMMARY

Each of the aspects disclosed herein may include one or more of the features described in connection with any of the other disclosed aspects.

The present disclosure includes a medical device comprising a shaft extending to a distal end and a pressure sensor assembly incorporated into the shaft. The pressure sensor assembly may include a micro-electromechanical system (MEMS) chip, a material, and a housing defining a chamber. The housing may include a first body coupled to a second body, e.g., wherein the first body may define at least one opening in fluid communication with the chamber. The MEMS chip may include a diaphragm. In some aspects, the material may be within the chamber and the opening, and may at least partially cover the diaphragm. The material may have an elasticity or viscosity that permits transfer of force therethrough. In some examples, the pressure sensor assembly may be configured to measure pressure, optionally both pressure and temperature, external to the medical device.

In some examples, the material may include a pigment that inhibits passage of electromagnetic radiation therethrough. Optionally, the material may comprise silicone or polyurethane. The material may completely cover the diaphragm. In some aspects, the MEMS chip may be suspended into the material. Further, for example, the material may fill the opening and may be directly exposed to an environment surrounding the medical device. In some examples, the chamber and the opening may be filled with the material. The opening may be a first opening aligned with the diaphragm, and optionally the first body may define a second opening in fluid communication with the chamber and the first opening. As mentioned above and elsewhere herein, the pressure sensor assembly may be configured to measure temperature external to the medical device. In some examples, the MEMS chip may be fixed to the second body. The MEMS chip may include a first material and the housing may include a second material different from the first material. A coefficient of thermal expansion of the first material may be approximately the same as a coefficient of thermal expansion of the second material.

In some aspects, the second body may include an insulating material and a plurality of passages. For example, each passage may house a conductor that electrically connects the MEMS chip to a corresponding electrical conductor extending proximally through the shaft. The insulating material may comprise a ceramic, for example. The plurality of passages may include three passages and the pressure sensor assembly may be electrically coupled to three electrical conductors. In some examples, the plurality of passages may include four passages and the pressure sensor assembly may be electrically coupled to four electrical conductors.

The present disclosure also includes a pressure sensor assembly having some or all of the features mentioned above and/or elsewhere herein. For example, the pressure sensor assembly may include a housing, a MEMS chip, and a material at least partially covering the MEMS chip. The housing may define a chamber and may include a first body coupled to a second body. The first body may define at least one opening in fluid communication with the chamber with the chamber. The MEMS chip may be within the chamber and may include a diaphragm. The diaphragm may be at least partially aligned with the opening. The material may have an elasticity or viscosity that permits transfer of force therethrough. The pressure sensor assembly may be configured to measure pressure externally to the pressure sensor assembly. Optionally, the pressure sensor assembly may be configured to measure both pressure and temperature externally to the pressure sensor assembly. In some examples, the material may comprise silicone or polyurethane. The opening may be a first opening and the first body may define a second opening in fluid communication with the chamber and the first opening. In some aspects, the diaphragm may be aligned with the first opening. Additionally or alternatively, the material may fill the chamber.

The present disclosure also includes a medical device comprising a shaft and a pressure sensor assembly incorporated into a distal portion of the shaft, wherein the pressure sensor assembly includes a housing, a MEMS chip, and a material. The housing may define a chamber and at least one opening in fluid communication with the chamber. The MEMS chip may be within the chamber and may include a diaphragm. The material may cover one or more electrical conduits of the pressure sensor assembly. In some examples, an end cap at a distal end of the shaft may define a channel through an exterior wall of the end cap. For example, a proximal end of the end cap may include a slot configured to receive the pressure sensor assembly. The pressure sensor assembly may be positioned within the slot, e.g., so that the opening is exposed to an environment surrounding the end cap. The pressure sensor assembly may be configured to generate an electrical signal based on a pressure applied to the material.

### BRIEF DESCRIPTION OF THE FIGURES

The accompanying drawings, which are incorporated in and constitute a part of this application, illustrate exemplary aspects that, together with the written descriptions, serve to explain the principles of this disclosure. Each figure depicts one or more exemplary aspects according to this disclosure, as follows:

FIG. 1 depicts an exemplary medical system and medical device according to some aspects of the present disclosure.

FIG. 2 depicts a perspective view of a distal portion of the exemplary medical device of FIG. 1.

FIG. 3A depicts a partial cutaway view of the distal portion of the exemplary medical device of FIG. 1.

FIG. 3B depicts a cross-sectional view of the distal portion of the medical device of FIG. 1.

FIGS. 4A-4D and 5 depict a pressure sensor assembly of the medical device of FIG. 1.

FIG. 6 depicts a substrate used in manufacturing the pressure sensor assembly of FIGS. 4A-4D and 5.

### DETAILED DESCRIPTION

Particular aspects of the present disclosure are described in greater detail below. The terms and definitions provided herein control, if in conflict with terms and/or definitions incorporated by reference. The term “distal” refers to a portion farthest away from a user when introducing a device into a subject (e.g., patient). By contrast, the term “proximal” refers to a portion closest to the user when placing the device into the subject. Proximal and distal directions are labeled with arrows marked “P” and “D,” respectively, throughout various figures.

As used herein, the terms “comprises,” “comprising,” “including,” “includes,” “having,” “has,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Relative terms such as “about,” “substantially,” and “approximately,” etc., are used to indicate a possible variation of ±10% of the stated numeric value or range.

Although ureteroscopes are referenced herein, it will be appreciated that the disclosure encompasses various medical devices that may be inserted into a body of a subject, such as endoscopes, duodenoscopes, gastroscopes, endoscopic ultrasonography (“EUS”) scopes, colonoscopes, bronchoscopes, laparoscopes, arthroscopes, cystoscopes, aspiration scopes, sheaths, or catheters.

The present disclosure includes medical devices comprising a sensor, e.g., a micro-electromechanical system (“MEMS”) assembly, such as a pressure sensor assembly, useful for therapeutic and/or diagnostic procedures. The MEMS assemblies herein may provide a combination of mechanical and electrical or electronic functionality such as, e.g., one or more sensors, chips (e.g., integrated circuit chip(s)) and/or other circuitry, and/or structural features. For example, the MEMS assembly may collect information regarding surrounding anatomy, e.g., a target organ, of a subject to assist a medical professional to determine an appropriate treatment.

In some examples of the present disclosure, the MEMS assembly may be integrated into or disposed on or within a distal portion of a medical device, such as a ureteroscope. The MEMS assembly may include a sensor. The sensor may be utilized, for example, to provide data associated with a therapeutic or diagnostic procedure performed inside the subject's body. As mentioned above, the MEMS assembly may be subjected to a variety of external forces that may damage the sensor and/or other components or aspects of the MEMS assembly. For example, the MEMS assembly may be subjected to external forces during insertion, removal and/or use. The forces may be a result of the MEMS assembly colliding with, or abutting against, the subject's tissue. The forces may also be a result of a lithotripsy procedure or other medical procedure that produces energy, e.g., shockwaves. Damage to the MEMS assembly may result in increased procedural times and/or decreased accuracy of measurements. Aspects of the present disclosure may include features to further protect the MEMS assembly. The scope of the present disclosure, however, is defined by the attached claims and not the ability to solve a specific problem.

According to some aspects of the present disclosure, a medical device may include a MEMS assembly (e.g., a pressure sensor assembly) and features useful for inhibiting or preventing damage thereto, among other aspects. In an aspect, the medical device may be part of a medical system. The medical device may include a handle and a shaft operably coupled thereto (e.g., extending from the handle), the shaft having a MEMS assembly incorporated within a distal end portion of the shaft.

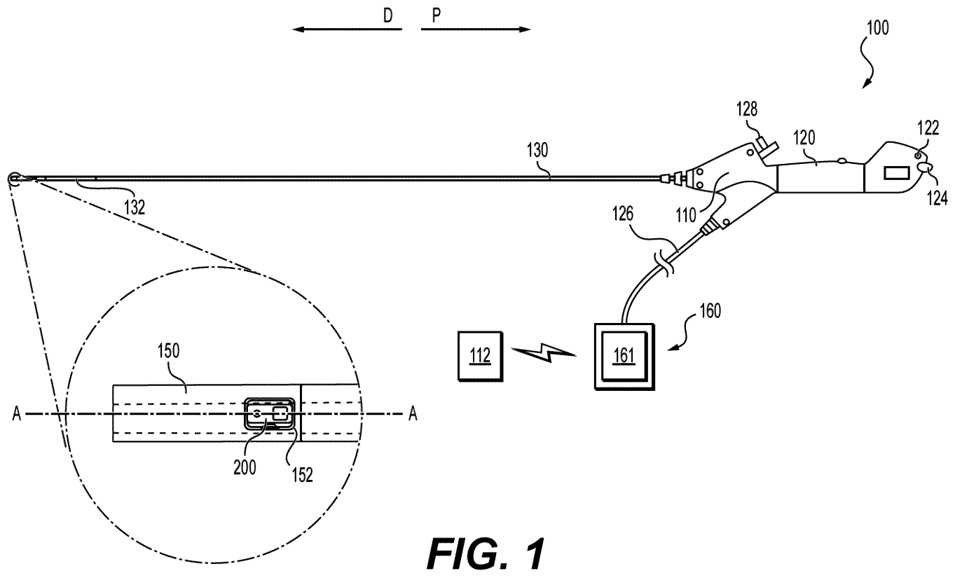

An exemplary medical system 100 is shown in FIG. 1. System 100 comprises a medical device 110, e.g., a scope, which may be operably coupled to equipment 160 supporting the medical device 110. For example, medical device 110 may include a bronchoscope, duodenoscope, endoscope, colonoscope, ureteroscope, etc. In this example, medical device 110 includes a handle 120 with at least one actuator, e.g., a first actuator 122 and a second actuator 124, a port 128, and a shaft 130 with a steerable portion 132. Shaft 130 may define a central longitudinal axis (axis A in FIG. 1).

Actuators 122, 124 may be configured to receive user input and transmit the user input to control movement of shaft 130. Each actuator 122, 124 may include a lever, knob, slider, joystick, button, or other suitable mechanism. For example, first actuator 122 may include a lever configured to articulate steerable portion 132, e.g., via one or more pull wires within shaft 130, and second actuator 124 may include a button configured to actuate and/or control other aspects of medical device 110, e.g., turning on/off a light source (e.g., light source 146) and/or capturing images via an imager (e.g., imager 142). Steerable portion 132 of shaft 130 may include a first pull wire with an end portion 125a and a second pull wire with an end portion 125b (FIG. 3B). End portions 125a, 125b may include ferrules to assist with securing the pull wires to shaft 130. In some examples, the first pull wire and second pull wire may be positioned opposite each other relative to the longitudinal axis of shaft 130.

Equipment 160 may be configured to supply medical device 110 with vacuum/suction, fluid (e.g., liquid, air), and/or power via umbilicus 126. Equipment 160 may include a processor 161, e.g., operable with medical device 110. For example, processor 161 may help generate a visual representation of image data and/or transmit the visual representation to one or more interface devices, e.g., a display 112. Display 112 may include, e.g., a monitor, touch-screen display, etc., capable of displaying images captured using medical device 110. While FIG. 1 shows processor 161 coupled to medical device 110 by umbilicus 126, in other examples, processor 161 may be incorporated into medical device 110. For example, processor 161 may be included in handle 120.

Port 128 may include one or more openings in communication with a working channel 134 (FIG. 3B) of shaft 130. While port 128 is illustrated in this example on a distal portion of handle 120, port 128 may disposed on or incorporated to other portions of handle 120. A suitable accessory instrument or tool, e.g., a fiber (e.g., laser fiber), grasper, retrieval device, etc., may be inserted through port 128 and moved distally through shaft 130 via working channel 134. Shaft 130 may further include one or more lumen(s) for receiving pull wires and/or other wiring, cables, and/or fluidics tubing.

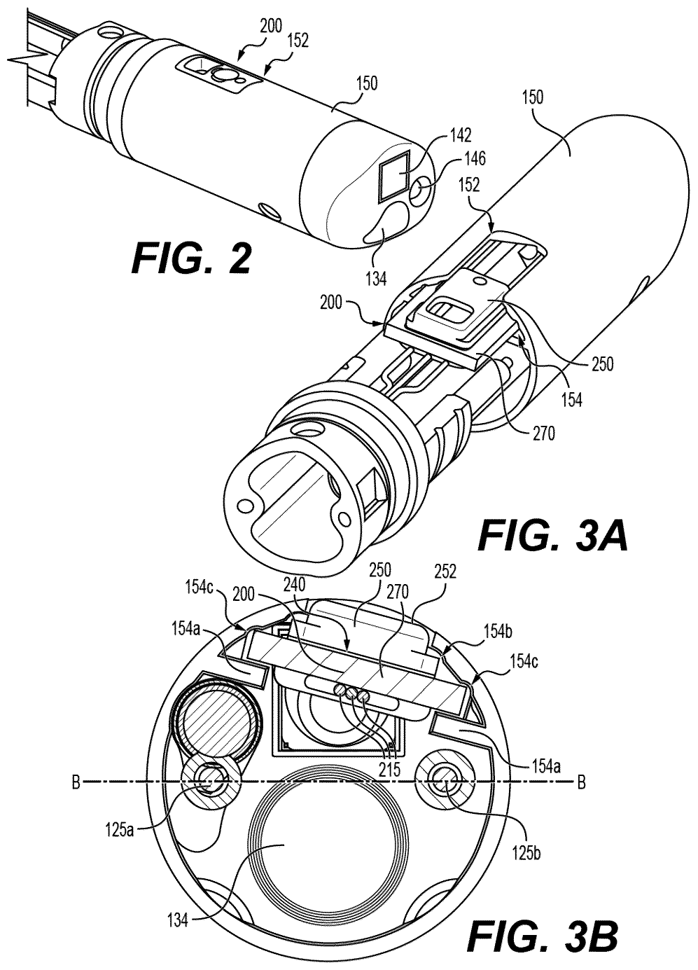

As shown in FIG. 2, the distal end of shaft 130 may include a distal opening of working channel 134, imager 142 (e.g., camera, fiber optics, or other imaging device), and a light source 146 (e.g., light-emitting diode (LED) or plastic optical fiber (POF) device). In some examples, the distal end of shaft 130 may include a cap 150 that includes electronic components such as imager 142 and light source 146. In some examples, imager 142 may include a camera. In some examples, imager 142 may be in communication with a sensor or other device within shaft 130 or handle 120.

Cap 150 may comprise a metal, metal alloy, polymer (e.g., plastic) or combination thereof. Cap 150 may include an opening 152, e.g., on a side surface and/or exterior wall of cap 150. Opening 152 may be proximate the distal end of shaft 130 and proximal to the distalmost end of shaft 130 as shown in FIGS. 1-3A. In other examples, at least part of opening 152 may be disposed on the distalmost face of shaft 130. Opening 152 may be aligned with at least a portion of a sensor, e.g., a MEMS assembly such as a pressure sensor assembly, to provide fluid communication between the sensor and the surrounding environment for measuring environmental parameters such as pressure.

FIGS. 3A and 3B depict a perspective view and a back view, respectively, of a portion of cap 150 and MEMS assembly, e.g., pressure sensor assembly 200. Pressure sensor assembly 200 may include a MEMS chip 210 that includes one or more sensors configured to measure one or more parameters such as pressure, temperature, humidity, position, light, and/or pH. For example, the one or more sensors of MEMS chip 210 may include sensor 220, e.g., a pressure sensor. In at least one example, sensor 220 is configured to measure both pressure and temperature. In at least one example, the one or more sensors of MEMS chip 210 may include a first sensor configured to measure pressure and a second sensor configured to measure temperature.

Sensor 220 may include a diaphragm 222 sensitive to pressure changes, e.g., to measure pressure in the environment around medical device 110. Exemplary materials suitable for diaphragm 222 include, but are not limited to, silicon, silicon dioxide (SiO₂), silicon nitride (Si₃N₄), silicon carbide (SiC), polysilicon, polyimide, aluminum nitride (AIN), metals (aluminum, titanium, etc.), piezoelectric materials (e.g., lead zirconate titanate (PZT), polymers (e.g., polydimethylsiloxane (PDMS)), graphene, and glass.

In other examples, diaphragm 222 may be formed from a conductive material. Diaphragm 222 may have a thickness to permit pressure measurements while avoiding cracking or breakage due to sudden changes in pressure. Generally, a greater thickness of diaphragm 222 may result in higher durability and lower thickness may result in higher sensitivity. Diaphragm 222 may be integrated with MEMS chip 210, e.g., such that the surface of diaphragm 222 is flush or approximately flush with the surface of MEMS chip 210. For example, sensor 220 may be within a cavity 224 of MEMS chip 210. When force is applied to the surface of diaphragm 222 according to the pressure around medical device 110, sensor 220 may measure pressure by the degree to which diaphragm 222 flexes into cavity 224. For example, sensor 220 may generate an electrical signal with a larger voltage when a larger pressure is applied to diaphragm 222. Conversely, sensor 220 may generate an electrical signal with a smaller voltage when a smaller pressure is applied to diaphragm 222.

Processor 161 may be configured to calculate the amount of pressure applied to diaphragm 222 based on the electrical signal generated by sensor 220. During an exemplary medical procedure, when sensor assembly 200 is in fluid communication with a bodily lumen or cavity (e.g., organ) of a patient, an internal pressure of the bodily lumen or cavity may cause diaphragm 222 to flex into cavity 224; and sensor 220 may generate an electrical signal based on the depth that diaphragm 222 flexes into cavity 224. Processor 161 may calculate the internal pressure of the bodily lumen based on the electrical signal and transmit the data for displaying the calculated internal pressure on display 112. In an exemplary procedure, pressure sensor assembly 200 may measure pressure within a kidney of the patient. Medical system 100 and pressure sensor assembly 200 are not limited to use within kidneys and may be used within any bodily lumen or cavity of a patient. Processor 161 may be calibrated before use by exposing pressure sensor assembly 200 to a controlled external environment of a known pressure.

Pressure sensor assembly 200 may include a housing 240 including a first body 250 and a second body 270. In addition to MEMS chip 210, pressure sensor assembly 200 may include one or more electrical conduits 212, conductive passages 213, first conductive pads 214a, second conductive pads 214b, and/or electrical conductors 215 (including but not limited to wires). In some examples, MEMS chip 210 defines or otherwise includes the electrical conduit(s) 212. MEMS chip 210 and electrical conduit(s) 212 may be within chamber 202. Electrical conduit(s) 212 may be electrically coupled to MEMS chip 210. In some aspects of the present disclosure, electrical conduit(s) 212 and/or conductive pads 214a, 214b may be omitted. For example, when conductive pads 214a, 214b are omitted, electrical conductor(s) 215 may be directly connected to conductive passage(s) 213 and electrical conduit(s) 212 may be directly connected to conductive passage(s) 213. In some examples, electrical conductor(s) 215 may extend through conductive passage(s) 213 and be directly connected to MEMS chip 210.

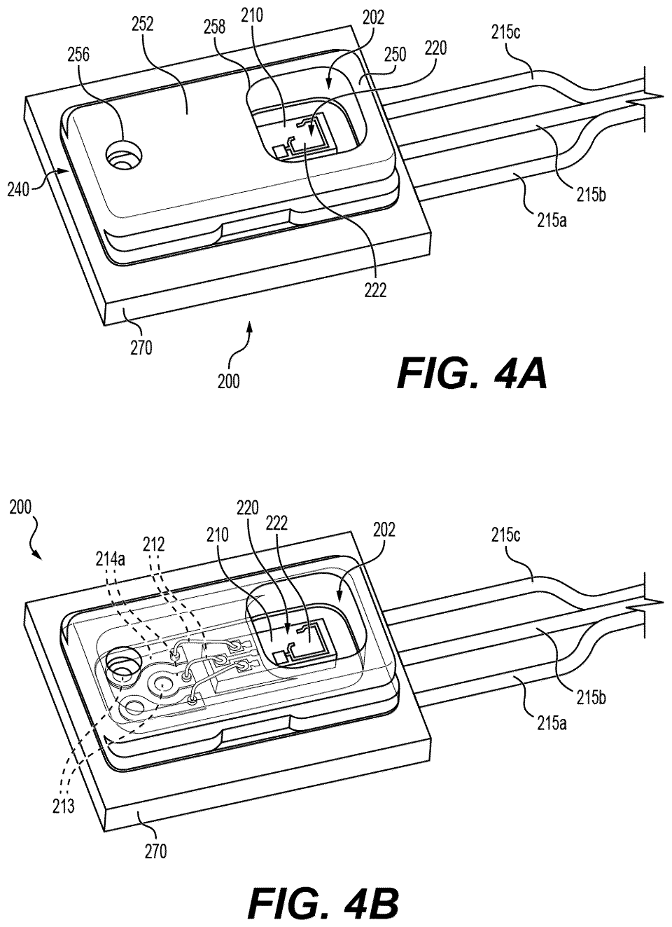

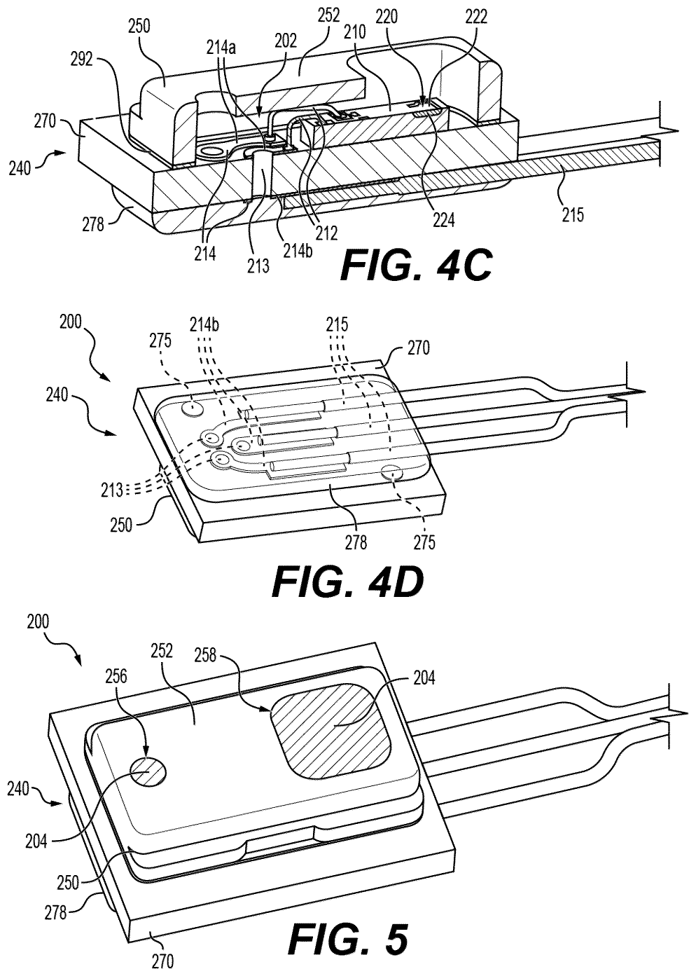

In some examples, electrical conductor(s) 215 may include three electrical conductors 215 including a first conductor 215a, a second conductor 215b, and a third conductor 215c (as seen in FIGS. 4A-4B). In other examples, electrical conductor(s) 215 may include three or more conductors 215, for example, electrical conductor(s) 215 may include four conductors 215. First conductor 215a may be a positive side output and third conductor 215c may be a negative side output. Second conductor 215b may be configured to supply voltage to MEMS chip 210. Processor 161 may be configured to compare voltages of first conductor 215a and third conductor 215c to determine pressure based on the compared voltages. As described above, processor 161 may calculate pressure on diaphragm 222 by comparing the voltages of first conductor 215a and third conductor 215c. MEMS chip 210 may include appropriate electrical connections and electrical elements to include a half-whetstone bridge. In some examples, electrical conductor(s) 215 may include a fourth conductor connected to ground for a full whetstone bridge. In such cases, second body 270 may include four conductive passages 213 to accommodate the electrical conductors 215. At least a portion of the electrical elements and connections of MEMS chip 210 forming the half-whetstone bridge or full whetstone bridge may be proximate diaphragm 222 and may be integrated within MEMS chip 210.

As mentioned above, pressure sensor assembly 200 may include first body 250. First body 250 may comprise a liquid crystal polymer (LCP), a polymer such as polyetheretherketone (PEEK) or polycarbonate, other nonconductive, thermoplastic materials, or; conductive resin; metal; and ceramic. First body 250 may include a rectangular prism shape as shown, however, this is merely exemplary and first body 250 may include other shapes. Second body 270 may have a coefficient of thermal expansion that is the same or about the same as the thermal expansion of MEMS chip 210. Second body 270 may include an insulating material. For example, the material of second body 270 may include one or more of ceramic substrate, flame retardant-4 (FR-4), LCP, or polymer (e.g., polyimide). Exemplary ceramics include, but are not limited to, silicon nitride, aluminum nitride and alumina. Optionally, a radially inward surface of first body 250 or a surface of second body 270 may include an adhesive layer 292.

As seen in at least FIGS. 4B and 4C, housing 240 may define a chamber 202 sized and shaped to contain MEMS chip 210 and one or more electrical conduits 212 electrically coupled to MEMS chip 210. Each electrical conduit 212 may be connected to a surface of conductive pad(s) 214a. Conductive pad(s) 214a, 214b may be adhered to and/or positioned on a surface of second body 270. In some examples, an entire inward-facing surface of MEMS chip 210 may be fixed to, adhered to, or contact second body 270. In some examples, only a portion of MEMS chip 210 is fixed to, adhered to, or otherwise contacts second body 270. For example, a portion of the inward-facing surface of MEMS chip 210 below the connections to electrical conduits 212 may be adhered to second body 270 while the remainder of the inward-facing surface of MEMS chip 210 is not adhered to second body 270.

Chamber 202 may include at least one opening, e.g., permitting introduction of a material therein to at least partially or completely fill chamber 202. In the example illustrated in FIGS. 4A-4D and 5, chamber 202 includes two openings, shown as a first opening 256 and a second opening 258 in surface 252 of first body 250. Surface 252 may be a radially outward-most surface of first body 250. First opening 256 as shown is smaller than second opening 258, but in other examples, the openings 256, 258 may be about the same size. According to some aspects, first opening 256 and second opening 258 may be sized sufficiently large to mitigate or prevent thermal hysteresis but sufficiently small to mitigate or prevent material 204 from exiting chamber 202 via one of openings 256, 258. MEMS chip 210 may be positioned within chamber 202 such that diaphragm 222 is generally aligned with second opening 258. According to some aspects, diaphragm 222 may be positioned within chamber 202 so that it is not aligned with second opening 258.

As seen in at least FIGS. 4C and 4D, second body 270 may define one or more conductive passages 213 therethrough. For example, second body 270 may define three passages 213. Passage(s) 213 may be filled with a conductive material or otherwise include a conductor therein, e.g., to provide electrical contact between conductive pads 214a, 214b. . Optionally, passage(s) 213 may include a conductive liner covering an internal wall of each passage 213, e.g., providing a conductive lumen. Conductive pad(s) 214a, 214b may be adhered to or otherwise positioned on second body 270. An exposed portion of a distal end of one or more electrical conductors 215 may be electrically coupled to/positioned on a surface of one or more conductive pads 214b. Similarly, an end of one or more electrical conduits 212 may be electrically coupled to/positioned on a surface of one or more conductive pads 214a and another end of one or more electrical conduits 212 may be electrically coupled to MEMS chip 210. Second body 270 may include one or more fiducial markers 275, e.g., to facilitate manufacturing by automated processing through machine vision. Pressure sensor assembly 200 may include an equal number of electrical conduits(s) 212, passage(s) 213, conductive pads 214a, conductive pads 214b, and electrical conductor(s) 215. For example, in examples in which second body 270 defines four passages 213, pressure sensor assembly 200 may include four electrical conduits 212, four conductive pads 214a, four conductive pads 214b, and four electrical conductors 215.

Still referring to FIGS. 4C and 4D, second body 270 may include a coating 278 over one or more of conductive pad(s) 214b, an end of passage(s) 213, and/or electrical conductor(s) 215. Coating 278 may comprise a waterproof, nonconductive, and/or adhesive material. Coating 278 may include adhesive such as, e.g., epoxy, acrylate (e.g., cyanoacrylate), ultra-violet (UV)-cured adhesive, conformal coating, and/or other hydrophobic materials. Coating 278 may provide waterproofing and/or strain-relief at conductive interfaces of passage(s) 213, conductive pad(s) 214b, and/or electrical conductor(s) 215.

As shown in FIG. 5, at least a portion or all of chamber 202 may be filled with a material, such as an elastic and/or viscous material 204. Material 204 may completely cover MEMS chip 210 and/or diaphragm 222. Material 204 may comprise, for example, silicone (e.g., silicone gel), epoxy or other adhesive, gelatin, oil (e.g., mineral oil), styrenic block copolymer (e.g., elastomers manufactured by Kraton Polymers), thermoplastic material (e.g., permagel), polyurethane (e.g., aromatic polyurethane such as Tecothane™ or a thermoplastic silicone polycarbonate polyurethane such as Carbosil®), and/or polycarbonate-based silicone elastomers (e.g., ChronoSil®). Material 204 may be non-conductive. In some aspects of the present disclosure, material 204 may comprise a pigment or dye, such that material 204 blocks or substantially limits the amount of electromagnetic radiation reaching diaphragm 222 and/or MEMS chip 210 through material 204, e.g., to protect diaphragm 222 and/or MEMS chip 210 from damage. In some examples, material 204 comprises a black pigment or dye. In some examples, about 5% by weight to about 20% by weight of material 204 may comprise a pigment or dye.

Material 204 may be cured, e.g., with heat during manufacturing, electromagnetic radiation (e.g., UV light), or humidity. Material 204 may have a relatively high viscosity and/or relatively low durometer value, e.g., such that material 204 remains in place within chamber 202. Material 204 may have a viscosity of about 25 Pa·s to about 10000 Pa·s at 25° C. In some examples, material 204 may have a viscosity of about 25 Pa·s to about 7000 Pa·s at 25° C. Material 204 may have a durometer Shore oo value ranging from 0 to about 95. In some examples, material 204 may have a durometer Shore A value ranging from 0 to about 70. The elasticity and/or viscosity of material 204 may facilitate transfer of force to diaphragm 222 to measure pressure. Material 204 may assist in dampening shockwaves and/or other forces on sensor 220 to limit and/or prevent damage to diaphragm 222. For example, a lithotripsy procedure may include use of a laser to break up stones, generating energy in the form of shockwaves.

In some examples, MEMS chip 210 may be suspended within material 204 in chamber 202 such that MEMS chip 210 does not contact second body 270 or first body 250. However, it should be understood while MEMS chip 210 does not contact second body 270 or first body 250 in these examples, electrical conduits 212 may contact conductive pads 214a. When MEMS chip 210 is suspended within material 204, thermo-mechanical effects on diaphragm 222, or other components of MEMS chip 210, may be diminished and/or limited, e.g., promoting sensitivity of sensor 220.

In some examples, as described above, a portion of chamber 202 may include material 204 that fills less than an entirety of chamber 202. In such cases, MEMS chip 210 may rest on a surface of material 204 within chamber 202. Chamber 202 may include material 204 to a depth that material 204 fully contains, encapsulates, and/or covers electrical conduits 212 in order to electrically isolate conduits 212 from the surrounding environment and/or fluid within chamber 202.

According to some aspects of the present disclosure, surface 252 of first body 250 may include a coating or film. The coating or film may cover surface 252 and the outermost surface of material 204. In other words, the coating or film may cover openings 256, 258. The coating or film may reduce tackiness/stickiness of material 204 flush with surface 252. Further, the coating or film may help to prevent material 204 from exiting chamber 202 via openings 256, 258. The coating or film may be hydrophilic, e.g., comprising a hydrophilic material. The hydrophilic material may help eliminate or reduce residual gas (e.g., air bubbles) within chamber 202 and/or material 204 or at a surface of material 204 which may improve the accuracy of sensor 220. For example, the hydrophilic material may reduce gas bubbles at the outermost surface of material 204. The coating or film may comprise, for example, polyethylene glycol (PEG), lauryl PEG-8 dimethicone (Silsurf®), polyurethane, biodurable aliphatic polycarbonate-based thermoplastic urethane, biodurable aromatic polycarbonate-based thermoplastic urethanes, ethyltriacetoxysilane, methyltriacetoxysilane, dibutyltin diaurate, hexamethyldisiloxane, trifluoroprophylmethylsiloxane, dimethylisiloxane, polydimethylsiloxane, sodium lauryl sulfate, ethylene oxide/propylene oxide block copolymer (e.g., Synperonic® F 108), octyl phenol ethoxylate (e.g., Triton X100), mineral oil, surfactants, silicone dispersions, silicone oils, and/or electrospun material(s). In some examples, the coating or film may comprise a plasma-treated polymer or other plasma-treated material. Plasma treatment may reduce surface energy of material 204 to reduce gas bubbles within material 204 or at a surface of material 204 (e.g., the outermost surface of material 204). In some examples, the coating or film be plasma treated (e.g., comprising plasma-treated material(s) and/or may have hydrophilic properties to reduce gas bubbles within material 204 or at a surface of material 204. In some examples, the coating or film may comprise a metal or metal alloy.

A distal end portion of shaft 130, e.g., cap 150, may include a slot 154 configured to receive pressure sensor assembly 200. For example, a proximal end of cap 150 may include slot 154. Slot 154 may be defined by one or more protrusions 154a extending from an inner surface of cap 150. In some examples, slot 154 may be configured to secure pressure sensor assembly 200 at an angle such that a midpoint of second body 270 is offset from a plane than includes the longitudinal axis of shaft 130 and a central axis of working channel 134. Protrusions 154a may extend at an angle relative to axis B shown in FIG. 3B. The angle of protrusions 154a may be transverse to axis B. In other examples, slot 154 and pressure sensor assembly 200 may extend along a plane parallel or perpendicular to axis B. Slot 154 may include notches 154b configured to receive edges of first body 250 and notches 154c configured to receive edges of second body 270.

When pressure sensor assembly 200 is received within slot 154, surface 252 of first body 250 may be positioned within opening 152 of end cap 150. In some examples, first body 250 may be received within opening 152 such that surface 252 is approximately flush an outer surface of cap 150. Opening 152 may be partially or completely aligned (e.g., generally aligned) with one or more of openings 256, 258 such that openings one or more 256, 258 may be exposed to the external environment during use. In other examples, surface 252 may be radially outward or radially inward of the outer surface of cap 150. Optionally, an adhesive may be applied over surface 252 of first body 250, e.g., to protect pressure sensor assembly 200 and/or prevent fluids from entering end cap 150.

Medical device 110, described herein, may be used during an exemplary medical procedure. Prior to the procedure, pressure sensor assembly 200 may be calibrated, e.g., in a controlled environment with known variables such as a known pressure and temperature. During the procedure, an operator (e.g., medical professional) may navigate the distal end of shaft 130 through a bodily lumen or cavity and proximate a target site. For example, the target site may be in the kidney or other organ. After reaching the target site, sensor 220, by its exposure to the environment around medical device 110, may be used to measure pressure at the target site. For example, pressure of the bodily lumen or cavity may be transferred to diaphragm 222, e.g., through material 204. Sensor 220 may generate an electrical signal based on movement of diaphragm 222. Processor 161 may calculate pressure based on the response by diaphragm 222, e.g., communicated to processor 161 via electrical conductors 215 as discussed above. Additionally or alternatively, sensor 220 may be configured to determine pressure based on the response by diaphragm 222 to pressure of the external environment, e.g., at a target site. Sensor 220 may determine temperature based on a bridge resistance of the half or full whetstone bridge of MEMS chip 210. For example, temperature may be proportional to bridge resistance and/or resistance of one or more electrical components of the half or full bridge. After processor 161 has determined the pressure and/or temperature at the target site, processor 161 may communicate with display 112 to display the pressure and/or temperature measurements on display 112. During the procedure, processor 161 may be configured to continuously and/or intermittently calculate the pressure and/or temperature at the target site for display on display 112.

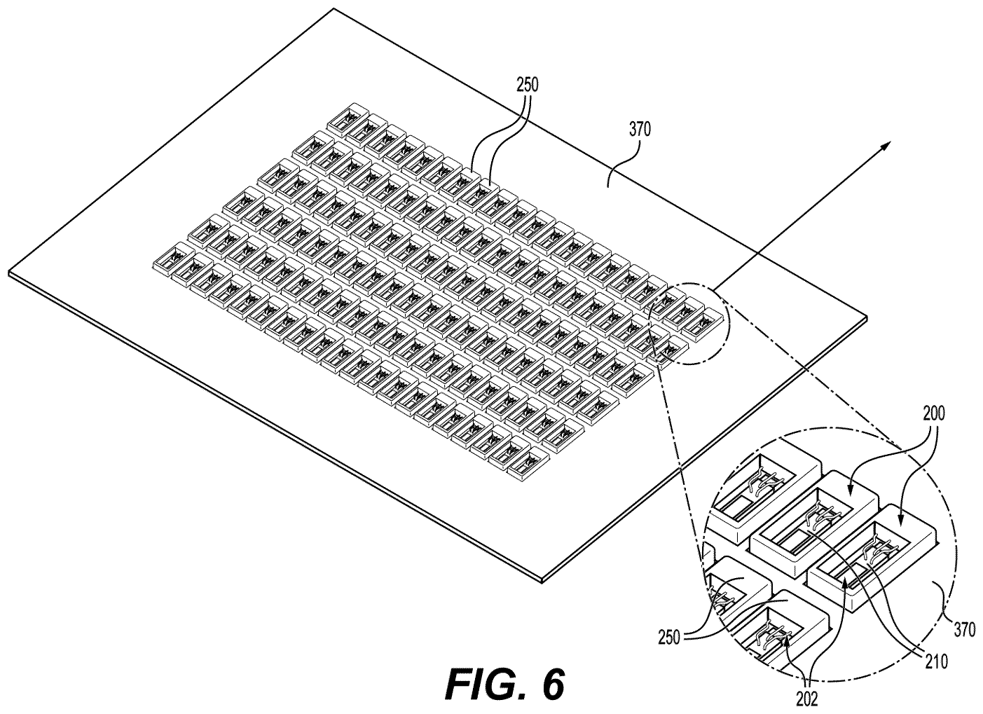

Pressure sensor assembly 200 may be manufactured using a substrate, and cutting individual units from a common substrate. FIG. 6 depicts a plurality of partially assembled pressure sensor assemblies 200. During manufacturing, a plurality of first bodies 250 may be positioned on a substrate 370 that when cut corresponds to second body 270 of each pressure sensor assembly 200. Portions of pressure sensor assemblies 200 (e.g., surface 252 of first body 250) are omitted from FIG. 6 for illustrative purposes to highlight features relevant to manufacturing pressure sensor assembly 200.

During manufacturing, one or more fiducial markers 275 may be positioned on or integrated within substrate 370 to help a machine during manufacturing to correctly place components and perform tasks. For example, a first surface of substrate 370 that receives first body 250 and MEMS chip 210 may include one or more markers 275 (e.g., in each corner when substrate 370 has a rectangular shape) and may include one or more markers 275 to designate between each row and column of pressure sensor assemblies 200 (rows and columns of assemblies 200 are shown in FIG. 6). A second, opposite surface of substrate 370 that receives coating 278 and electrical conductors 215 may include one or more markers 275 for each one of the plurality of pressure sensor assemblies 200. FIG. 4D shows markers 275 used to arrange pressure sensor assemblies 200 during manufacturing. In addition to or in replacement of markers 275, other physical landmarks may be used (e.g., by a machine) during manufacturing to correctly orient features such as passages 213. It should be understood that although one machine is described as performing all of the steps in the following paragraphs, one or more different machines may be used to perform each step.

Further during manufacturing, the machine may apply an adhesive to a surface (e.g. the first surface) of substrate 370. The adhesive may be dissolvable when exposed to a fluid (e.g., water). The machine may utilize markers 275 to properly place the adhesive. After applying the adhesive, the machine may place or otherwise position MEMS chip 210 (e.g., the radially inward-facing surface of MEMS chip 210) on the adhesive. While placing MEMS chip 210, the machine may use fiducial markers 275 to correctly place MEMS chip 210. The adhesive may have a depth or thickness between substrate 370 and MEMS chip 210. According to some aspects of the present disclosure, MEMS chip 210 may be placed onto substrate 370 directly and the adhesive may be introduced to then adhere MEMS chip 210 to substrate 370.

Before or after placing MEMS chip 210, passages 213 may be provided through substrate 370. For example, the machine may drill through substrate to create passages 213. The machine may position and connect conductive pads 214a, 214b to each passage 213 as described above. When positioning and connecting conductive pads 214a, 214b, the machine may utilize a location or position of one or more of passages 213 to correctly position conductive pads 214, 214b. Alternatively, the machine may utilize fiducial markers 275.

After placing MEMS chip 210, the machine may provide electrical conduits 212 and then may connect electrical conduits 212 to conductive pads 214a and MEMS chip 210. Electrical conduits 212 may be sufficiently rigid such that each MEMS chip 210 is supported by one or more electrical conduits 212 and maintained in the corresponding chamber 202 after first bodies 250 are positioned. After connecting electrical conduits 212, the machine may apply a fluid to the adhesive to dissolve and remove the adhesive. According to some aspects of the present disclosure, the step of dissolving and removing the adhesive may be omitted and the adhesive may be left in place. According to some aspects of the present disclosure, a portion of the adhesive under or generally aligned with an end of electrical conduits 212 connected to MEMS chip 210 may be left in place while a remainder of the adhesive may be removed resulting in a portion of the radially inward-facing surface of MEMS chip 210 being angled away from substrate 370. As described above, after dissolving and removing the adhesive, electrical conduits 212 may be sufficiently rigid such that MEMS chip 210 is maintained in place. For instance, when MEMS chip 210 is placed on the adhesive, a distance between the radially inward-facing surface of MEMS chip 210 and substrate 370 may be maintained by electrical conduits 212 via conduits 212 supporting MEMS chip 210.

After placing MEMS chip 210 or after connecting electrical conduits 212, the machine may position each first body 250 in proper position relative to fiducial markers 275. Each first body 250 may be adhered or otherwise affixed to substrate 370. For example, a radially inward-facing surface of each first body 250 may include an adhesive layer to adhere first body 250 to substrate 370.

After placing first body 250, the machine may introduce material 204 through first opening 256 of each pressure sensor assembly 200 to at least partially or completely fill chamber 202, e.g., such that material 204 is flush with surface 252 of each first body 250 in the case of completely filling chamber 202. According to some aspects of the present disclosure, instead of material 204 being flush with surface 252 the material may include a concave or convex surface at or within openings 256, 258. Residual air from chamber 202 may be capable of escaping chamber 202 through second opening 258 during introduction of material 204 into first opening 256. In aspects where the adhesive used to adhere MEMS chip 210 to substrate 370 is not removed or partially removed, material 204 may additionally surround and/or encapsulate the adhesive. According to some aspects of the present disclosure, material 204 may partially fill chamber 202 only to a depth or amount sufficient to cover or coat electrical conduits 212 (e.g., to waterproof electrical conduits 212) and may leave diaphragm 222 uncovered by material 204. Other fluid, such as fluid from the surrounding environment, may apply pressure to diaphragm 222 indicative of the pressure of the surrounding environment. After or before material 204 has been cured, e.g., by application of heat or UV light, a film may be applied over surface 252 of first body 250 and over material 204 within openings 256, 258, e.g., to reduce stickiness/tackiness of material 204 and/or prevent material 204 from exiting chamber 202 via openings 256, 258.

Before or after introducing material 204 to fill chamber 202, substrate 370 may be divided (e.g., cut or otherwise separated) into a plurality of second bodies 270, each corresponding to an individual pressure sensor assembly 200. For example, the machine may cut substrate 370 into a plurality of second bodies 270 and may utilize fiducial markers 275 during cutting. The machine may utilize a laser to cut substrate 370.

After dividing substrate 370 into a plurality of second bodies 270, the machine may provide electrical conductors 215 and then position and connect ends of electrical conductors 215 to conductive pads 214b. The machine may utilize markers 275 and/or passages 213 to correctly position and connect electrical conductors 215 to conductive pads 214b. Further, after positioning electrical conductors 215, the machine may provide coating 278 and position coating 278 over one or more of conductive pad(s) 214b, an end of passage(s) 213, and/or electrical conductor(s) 215. The machine may utilize markers 275 and/or passages 213 to correctly place coating 278.

While principles of this disclosure are described herein with reference to illustrative examples for particular applications, it should be understood that the disclosure is not limited thereto. Those having ordinary skill in the art and access to the teachings provided herein will recognize additional modifications, applications, embodiments, and substitution of equivalents all fall within the scope of the features described herein. Accordingly, the claimed features are not to be considered as limited by the foregoing description.

What is claimed is:

1. A medical device comprising:
a shaft extending to a distal end; and
a pressure sensor assembly incorporated into the shaft, the pressure sensor assembly comprising:
a housing defining a chamber and including a first body coupled to a second body, the first body defining at least one opening in fluid communication with the chamber,
a MEMS chip including a diaphragm; and
a material within the chamber and the opening, the material at least partially covering the diaphragm and having an elasticity or viscosity that permits transfer of force therethrough;
wherein the pressure sensor assembly is configured to measure pressure external to the medical device.

2. The medical device of claim 1, wherein the material comprises a pigment that inhibits passage of electromagnetic radiation therethrough.

3. The medical device of claim 1, wherein the material comprises silicone or polyurethane.

4. The medical device of claim 1, wherein the material completely covers the diaphragm.

5. The medical device of claim 3, wherein the MEMS chip is suspended within the material.

6. The medical device of claim 5, wherein the material fills the opening and is directly exposed to an environment surrounding the medical device.

7. The medical device of claim 1, wherein the opening is a first opening aligned with the diaphragm and the first body defines a second opening in fluid communication with the chamber and the first opening.

8. The medical device of claim 1, wherein the second body comprises an insulating material and includes a plurality of passages, each passage housing a conductor that electrically connects the MEMS chip to a corresponding electrical conductor extending proximally through the shaft.

9. The medical device of claim 8, wherein the insulating material comprises a ceramic.

10. The medical device of claim 8, wherein the plurality of passages includes three passages, and the pressure sensor assembly is electrically coupled to three electrical conductors.

11. The medical device of claim 8, wherein the plurality of passages includes four passages, and the pressure sensor assembly is electrically coupled to four electrical conductors.

12. The medical device of claim 1, wherein the pressure sensor assembly is also configured to measure temperature external to the medical device.

13. The medical device of claim 1, wherein the chamber and the opening are filled with the material.

14. The medical device of claim 1, wherein the MEMS chip comprises a first material and the housing comprises a second material different from the first material, and wherein a coefficient of thermal expansion of the first material is approximately the same as a coefficient of thermal expansion of the second material.

15. The medical device of claim 1, wherein the MEMS chip is fixed to the second body.

16. A pressure sensor assembly comprising:
a housing defining a chamber and including a first body coupled to a second body, the first body defining at least one opening in fluid communication with the chamber;
a MEMS chip within the chamber and including a diaphragm at least partially aligned with the opening; and
a material at least partially covering the MEMS chip, the material having an elasticity or viscosity that permits transfer of force therethrough;
wherein the pressure sensor assembly is configured to measure pressure external to the pressure sensor assembly.

17. The pressure sensor assembly of claim 16, wherein the material comprises silicone or polyurethane.

18. The pressure sensor assembly of claim 16, wherein the opening is a first opening and the first body defines a second opening in fluid communication with the chamber and the first opening, the diaphragm being aligned with the first opening.

19. The pressure sensor assembly of claim 16, wherein the material fills the chamber.

20. A medical device comprising:
a shaft; and
a pressure sensor assembly incorporated into a distal portion of the shaft, the pressure sensor assembly comprising:
a housing defining a chamber and at least one opening in fluid communication with the chamber,
a MEMS chip within the chamber and including a diaphragm; and
a material covering one or more electrical conduits of the pressure sensor assembly;
wherein an end cap at a distal end of the shaft defines an opening through an exterior wall of the end cap;
wherein a proximal end of the end cap includes a slot;
wherein the pressure sensor assembly is positioned within the slot so that the at least one opening of the housing is exposed to an environment surrounding the end cap and aligned with the opening of the end cap;
wherein the pressure sensor assembly is configured to generate an electrical signal based on a pressure applied to the material.
